(19) United States
(12) Patent Application Publication
(10) Pub. No.: US 2025/0017843 A1
(43) Pub. Date: Jan. 16, 2025

(54) AMPHIPHILIC THICKENER FOR PERSONAL CARE

(71) Applicant: LG H&H CO., LTD., Seoul (KR)

(72) Inventors: Jun Seok YEOM, Seoul (KR); Woo Sun SHIM, Seoul (KR)

(73) Assignee: LG H&H CO., LTD., Seoul (KR)

(21) Appl. No.: 18/763,811

(22) Filed: Jul. 3, 2024

(30) Foreign Application Priority Data
Jul. 3, 2023 (KR) 10-2023-0085964
Nov. 28, 2023 (KR) 10-2023-0168298

(51) Int. Cl. A61K 8/895; A61K 8/06; A61K 8/81; A61Q 1/00; A61Q 17/04

(52) U.S. Cl. CPC A61K 8/895; A61K 8/064; A61K 8/8158; A61K 8/8188; A61Q 1/00; A61Q 17/04; A61K 2800/48; A61K 2800/596; A61K 2800/624; A61K 2800/654; A61K 2800/805

(57) ABSTRACT

The present invention relates to a thickener that may be used in cosmetics and the like, and more specifically, to a thickener that may provide moisture with less stickiness.

In the present invention, a thickener in the form of homogeneous fine particles whose surface is coated with a silicone polymer may be prepared using phase inversion temperature (PIT) polymerization while allowing the polymerization reaction to proceed in a uniform reverse phase emulsion with a small particle size. In addition, the thickener may be prepared using precipitation polymerization.

In addition, the spherical particle-shaped thickener prepared by the present invention has the advantage of high moisture retention due to the coated silicone polymer, which is the shell, while maintaining the shape of the particle even when swelling in moisture, and the spherical particles, whose surface is coated with the silicone polymer, have high dispersibility in an oil phase, which helps to maintain makeup, and the spherical polymer has the effect of reducing stickiness and staining.

### BACKGROUND

### 1. Field of the Invention

The present invention relates to a thickener that may be used in compositions or products for personal care such as cosmetics and the like, and more specifically, to a thickener that may provide moisture with less stickiness.

### 2. Discussion of Related Art

Since an important attribute when using makeup or sunscreen products is that the makeup or sunscreen products have high makeup or UV protection durability, so once applied, the makeup effect lasts for a long time without needing to correct or reapply. However, in order to increase makeup durability, since a water-in-oil (W/O) formulation is used, which has excellent water resistance against sweat and sebum resistance, there is a disadvantage in that it lacks moisture and makes a user feel dry. In addition, the volatile oil used for quick setting when applied to the skin reduces the formability of the formulation during product manufacturing, resulting in lower productivity.

To solve this problem, when the aqueous phase ratio of the formulation is increased, the stability of the formulation is impaired due to the nature of the water-in-oil type, and phase separation easily occurs, and when more emulsifiers are used to enhance the stability of the formulation, a vicious cycle of increased stickiness occurs. In addition, when a film former is used to increase makeup durability, stickiness and staining also increase.

Therefore, there is a need for technology that can increase the durability of makeup and UV protection while increasing moisture and improving stickiness and staining.

To solve this problem, the present inventors discovered that the feeling of moisture was increased by suppressing moisture evaporation from the core portion and at the same time, makeup durability, stickiness, and staining were improved due to silicone polymers in the shell portion, by coating the core part with a polymer that can absorb water and maintain viscosity, and chemically coating the shell part with a silicone polymer (based on the fine particle structure polymer of a previous study, Patent Registration No. 10-2531603), and completed the present invention.

### RELATED ART DOCUMENT

### [Patent Document]

Korea Patent Registration No. 10-2531603

### SUMMARY OF THE INVENTION

The present invention is directed to providing an amphiphilic thickener for personal care that may increase moisture and improve stickiness and staining.

The present invention is also directed to providing cosmetics including the amphiphilic thickener of the present invention with excellent cosmetic durability and UV protection durability.

In order to achieve the above objects, the amphiphilic thickener of the present invention includes a crosslinked polymer obtained by polymerizing a water-soluble monomer represented by the following Chemical Formula 1, a crosslinkable monomer, and a silicone-containing monomer represented by the following Chemical Formula 2:

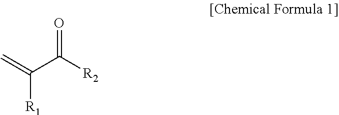

wherein
R₁ is —H, —CH₃, —CH₂CH₃, or —(CH₂)ₘCH₃, and here m=1 to 3,
R₂ is —OX, NH₂, or —NH—R₃—SO₃Y,
R₃ is —(CH₂)—, —(CH₂)₂—, —(CH₂)₃—, —(CH₂)₄—, —(CH₂)₂(CH₃)₂—, or —(CH₂)₃CH₃—, and
X and Y are H⁺, Na⁺, Li⁺, K⁺, or NH₃⁺ and may be the same,

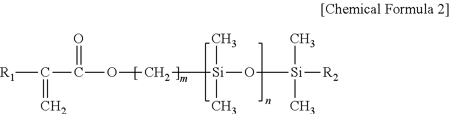

wherein
m is an integer from 1 to 5,
n is an integer from 10 to 5000,
R₁ is hydrogen or a C₁₋₃ alkyl group, and
R₂ is a hydroxy group, a C₁₋₃ alkyl group, or a vinyl group.

In one embodiment, in order to prepare the amphiphilic thickener, the present invention includes a method of preparing a crosslinked polymer obtained by dissolving a water-soluble monomer represented by Chemical Formula 1, a crosslinkable monomer, and the silicone-containing monomer represented by Chemical Formula 2, raising a temperature, and then adding a reaction initiator and performing polymerization.

In one embodiment, a method of preparing the amphiphilic thickener of the present invention may include: preparing an oil-in-water emulsion composition including an aqueous phase including a water-soluble monomer represented by Chemical Formula 1 and a crosslinkable monomer, and an oil phase including a silicone-containing monomer represented by Chemical Formula 2 and a non-polar organic solvent; preparing a water-in-oil reverse phase emulsion composition by raising a temperature to 60° C. or higher; and adding a reaction initiator and performing polymerization to prepare a crosslinked polymer.

In one embodiment, phase inversion temperature (PIT) polymerization may be used as a method of preparing the amphiphilic thickener.

In another embodiment, a method of preparing the amphiphilic thickener of the present invention may include: dissolving a water-soluble monomer represented by Chemical Formula 1, a crosslinkable monomer, and a silicone-containing monomer represented by Chemical Formula 2 in an alcohol-based solvent; raising a temperature to 60° C. or higher; and adding a reaction initiator and performing polymerization to prepare a crosslinked polymer. Precipitation polymerization may be used as a method of preparing the amphiphilic thickener.

In one embodiment, the method of preparing the amphiphilic thickener may further include obtaining the thickener as a solid, and for example, precipitation using a non-solvent of the crosslinked polymer may be used.

### BRIEF DESCRIPTION OF THE DRAWINGS

FIG. 1 is an exemplary diagram showing a method of preparing an amphiphilic thickener according to inverse emulsion polymerization in the present invention.

FIG. 2 is an exemplary diagram showing a method of preparing an amphiphilic thickener according to precipitation polymerization in the present invention.

FIG. 3 is a graph showing the results of confirming the moisture retention of the amphiphilic thickener according to the present invention.

FIG. 4 is a photo showing the results of confirming the staining of the makeup formulation to which the amphiphilic thickener according to the present invention was applied.

FIG. 5 is a graph showing the results of confirming the stickiness of a sunscreen formulation using the amphiphilic thickener according to the present invention.

### DETAILED DESCRIPTION OF EXEMPLARY EMBODIMENTS

However, since the present invention may be subject to various modifications and may have various forms, the specific embodiments and description described below are intended merely to aid the understanding of the present invention and are not intended to limit the present invention to a specific disclosed form. It should be understood that the scope of the present invention includes all modifications, equivalents, and alternatives falling within the spirit and scope of the present invention.

Hereinafter, the present invention will be described in more detail.

In the present invention, a “thickener” refers to a crosslinked polymer prepared according to the preparation method of the present invention, and may refer to a solution in which the crosslinked polymer is dispersed. In particular, the thickener may refer to a substance typically used to increase viscosity in compositions for personal care or products for personal care.

In one embodiment, the amphiphilic thickener of the present invention includes a crosslinked polymer obtained by polymerizing a water-soluble monomer represented by the following Chemical Formula 1, a crosslinkable monomer, and a silicone-containing monomer represented by the following Chemical Formula 2:

In one embodiment, the water-soluble monomer may be a compound of the following Chemical Formula 1.

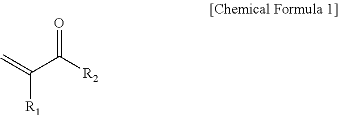

wherein
R₁ is —H, —CH₃, —CH₂CH₃, or —(CH₂)ₘCH₃, and here m=1 to 3,
R₂ is —OX, NH₂, or —NH—R₃—SO₃Y,
R₃ is —(CH₂)—, —(CH₂)₂—, —(CH₂)₃—, —(CH₂)₄—, —(CH₂)₂(CH₃)₂—, or —(CH₂)₃CH₃—, and
X and Y are H⁺, Na⁺, Li⁺, K⁺, or NH₃⁺ and may be the same,

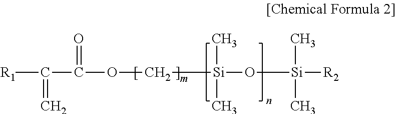

wherein
m is an integer from 1 to 5,
n is an integer from 10 to 5000,
R₁ is hydrogen or a C₁₋₃ alkyl group, and
R₂ is a hydroxyl group, a C₁₋₃ alkyl group, or a vinyl group.

In one embodiment, a method of preparing the amphiphilic thickener of the present invention may include: preparing an oil-in-water emulsion composition including an aqueous phase including a water-soluble monomer represented by Chemical Formula 1 and a crosslinkable monomer, and an oil phase including a silicone-containing monomer represented by Chemical Formula 2 and a non-polar organic solvent (step 1-1); preparing a water-in-oil reverse phase emulsion composition by raising a temperature to 60° C. or higher (step 1-2); and adding a reaction initiator and performing polymerization to prepare a crosslinked polymer (step 1-3).

In one embodiment, phase inversion temperature (PIT) polymerization may be used as a method of preparing the amphiphilic thickener. PIT polymerization may form an oil-in-water emulsion composition when the temperature is low, such as in step 1-1, which may then be converted to a water-in-oil reverse phase emulsion composition by increasing the temperature through step 1-2.

In the present invention, step 1-1 is a step of preparing an oil-in-water emulsion composition including an aqueous phase including a water-soluble monomer represented by the following Chemical Formula 1 and a crosslinkable monomer, and an oil phase including a silicone-containing monomer represented by the following Chemical Formula 2 and a non-polar organic solvent.

In the present invention, the aqueous phase in step 1-1 includes the water-soluble monomer represented by Chemical Formula 1 and the crosslinkable monomer.

In one embodiment, the water-soluble monomer represented by Chemical Formula 1 in step 1-1 may be at least one selected from the group consisting of acrylamidomethylpropane sulfonic acid (AMPS), acrylic acid, acrylamide, and polyethylene glycol acrylate.

Specifically, the water-soluble monomer represented by Chemical Formula 1 may be a compound represented by the following Chemical Formula 1a.

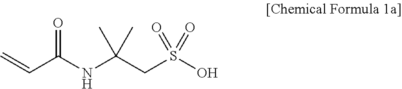

In the present invention, the content of the water-soluble monomer represented by Chemical Formula 1 in step 1-1 is not particularly limited, but for example, the water-soluble monomer may be included in an amount of 10 to 99% by weight, preferably 30 to 95% by weight, and more preferably 50 to 95% by weight, based on 100% by weight, which is the total sum of the water-soluble monomer represented by Chemical Formula 1, the crosslinkable monomer, and the silicone-containing monomer represented by Chemical Formula 2. Within the above range, the stability of the composition may be more stabilized and the stickiness improvement effect may be more excellent.

In one embodiment, the crosslinkable monomer in step 1-1 may be a compound having two or more acrylate groups, two or more acrylamide groups, or two or more vinyl groups. As the crosslinkable monomer, for example, one or more selected from the group consisting of trimethylolpropane ethoxylate triacrylate (TMPETA), trimethylolpropane triacrylate (TMPTA), methylenebisacrylamide, divinylsulfone, divinylbenzene, divinyl ether, divinylacetylene, polyglycol diacrylate, polyglycol triacrylate, and 4-arm-polyglycol tetraacrylate may be used.

In one embodiment, as the crosslinking monomer in step 1-1, more preferably, trimethylolpropane ethoxylate triacrylate (TMPETA), trimethylolpropane triacrylate (TMPTA), or a combination thereof may be used. The crosslinkable monomer is a compound having three or more crosslinking points and may improve the molecular weight of the crosslinked product, thereby improving viscosity.

In the present invention, the content of the crosslinkable monomer in step 1-1 is not particularly limited, but for example, the crosslinkable monomer may be included in an amount of 0.01 to 20% by weight, preferably 0.1 to 10% by weight, and more preferably 0.5 to 5.0% by weight, based on 100% by weight, which is the total sum of the water-soluble monomer represented by Chemical Formula 1, the crosslinkable monomer, and the silicone-containing monomer represented by Chemical Formula 2. Within the above range, the composition may have better stability or a better stickiness improvement effect.

In one embodiment, a solvent of the aqueous phase in step 1-1 may be water (distilled water).

In one embodiment, the pH of the aqueous phase in step 1-1 is not particularly limited, but may be, for example, 5 to 9. The pH may be adjusted using a common pH adjuster, for example, ammonia water.

In the present invention, the oil phase in step 1-1 may include a silicone-containing monomer represented by Chemical Formula 2 and an organic solvent.

In the present invention, the content of the silicone-containing monomer represented by Chemical Formula 2 in step 1-1 is not particularly limited, but for example, the silicone-containing monomer may be included in an amount of 10 to 70% by weight, preferably 5 to 60% by weight, and more preferably 10 to 50% by weight, based on 100% by weight, which is the total sum of the water-soluble monomer represented by Chemical Formula 1, the crosslinkable monomer, and the silicone-containing monomer represented by Chemical Formula 2. Within the above range, the composition may have better stability or a better stickiness improvement effect.

In one embodiment, the organic solvent may be a non-polar organic solvent, specifically, C₆₋₁₇ hydrocarbon oil, or C₆₋₁₇ straight chain saturated hydrocarbon oil. When the carbon number is outside the above range, production efficiency may be reduced in the polymerization reaction.

In one embodiment, heptane may be used as the non-polar organic solvent. Heptane has a boiling point of 98° C. and is not restricted in the polymerization process according to the present invention compared to other organic solvents. During the process, there is a risk that heat is generated due to an explosive reaction during the initiator input process, causing the temperature inside the reactor to rise by approximately 5 to 10° C. The method of slowly inputting the initiator to maintain homeostasis of the reaction temperature has the disadvantage of increasing the process time when applied to a mass production process, which increases production costs. In addition, when the temperature exceeds the boiling point, since the reactants bump into each other and the risk of the reactor exploding increases, the management of reaction heat is a very important factor during mass production. Therefore, in the present invention, by using heptane as an organic solvent, the thickener targeted by the present invention may be easily prepared without process restrictions.

In the present invention, the oil phase in step 1-1 may further include a surfactant.

In one embodiment, one or more surfactants may be used as the surfactant in step 1-1. A total HLB value of the surfactant may be 6 to 14, 7 to 10, or 8 to 9. At this time, the total HLB value may mean the HLB value of one surfactant when one surfactant is used, and may mean the sum of the HLB values of two or more surfactants when two or more surfactants are used. When the total HLB value is 6 or less, due to the strong hydrophobicity of the surfactant, since it is present as a water-in-oil type reverse phase emulsion at room temperature and is polymerized without a phase inversion process, the effect of reducing the size of the emulsion may not be sufficient. In addition, when the total HLB value is 14 or more, since the oil-in-water emulsion with the outer aqueous phase is maintained due to the strong hydrophilicity of the surfactant, and spherical particles are not formed, there is a concern that the amorphous state of the prepared thickener will increase.

In one embodiment, two types of surfactants may be used as the surfactant in step 1-1. In this case, a surfactant with an HLB value of 3 to 8 and a surfactant with an HLB value of 8 to 16 may be used in combination, or a surfactant with an HLB value of 5 to 7 and a surfactant with an HLB value of 9 to 11 may be used in combination.

In one embodiment, a combination of polyoxyethylene (3) oleyl ether (HLB=6.6) and polyoxyethylene (6) oleyl ether (HLB=9.6) may be used as the surfactant in step 1-1. Specifically, polyoxyethylene (3) oleyl ether (HLB=6.6) and polyoxyethylene (6) oleyl ether (HLB=9.6) may be used in combination at a weight ratio of 1:1, and here the HLB value may be 8.1.

Specifically, by an appropriate combination of two types of surfactants such as a surfactant having an HLB value of 3 to 8 and a surfactant having an HLB value of 8 to 16 or two types of surfactants such as a surfactant with an HLB value of 5 to 7 and a surfactant with an HLB value of 9 to 11, polymerization may be allowed to proceed in a uniform reverse phase emulsion with a small particle size using phase inversion temperature (PIT) polymerization, which converts an oil-in-water (o/w) emulsion phase at room temperature into a water-in-oil (w/o) emulsion phase above a certain temperature. Through this, in the present invention, a thickener in the form of homogeneous spherical fine particles may be prepared.

The thickener in the form of spherical fine particles polymerized in the above manner not only maintains the shape of the particles even when swelling in the moisture phase, but also has the advantage of a transparent appearance and high stability due to uniform particle dispersion.

In one embodiment, the content of the surfactant in step 1-1 is not particularly limited, but may vary depending on the type of organic solvent. Specifically, the content of the surfactant may be 5 to 30% by weight based on 100% by weight, which is the total sum of purified water, an organic solvent, a surfactant, a water-soluble monomer represented by Chemical Formula 1, a crosslinkable monomer, and a silicone-containing monomer represented by Chemical Formula 2. In the above content range, phase inversion of the emulsion is possible and PIT polymerization may be easily performed.

In one embodiment, a weight ratio of the aqueous phase and the oil phase in step 1-1 may be 30:70 to 70:30. Within the above range, the composition may be more stabilized or the stickiness improvement effect may be better.

In the present invention, step 1-2 is a step of preparing a water-in-oil reverse phase emulsion composition by raising the temperature to 60° C. or higher. In step 1-2, the temperature may be raised to 50° C. to 80° C., preferably 55° C. to 75° C., and when the temperature is raised to below 50° C., there is a problem that the phase transition of the emulsion is difficult.

In one embodiment, the emulsion prepared in step 1-1 of the present invention is an oil-in-water emulsion at room temperature, but the phase thereof may be inverted to a water-in-oil emulsion by step 1-2. The emulsion generated by the above phase inversion may have the effect of reducing the size of the emulsion and may have the effect of controlling the particle size of the polymer prepared in the step described later to be small.

In one embodiment, the reaction temperature in step 1-2 may be, for example, 62° C. or higher, 65° C. or higher, 70° C. or higher, or 75° C., and the upper limit may be 100° C. The thickener may be prepared more stably in the above temperature range.

In the present invention, step 1-3 is a step of adding a reaction initiator and performing polymerization to prepare a crosslinked polymer.

In one embodiment, the type of reaction initiator in step 1-3 is a typical radical polymerization initiator, is not particularly limited, but for example, may be selected from the group consisting of peroxides and azo compounds. As the peroxide initiator, benzoyl peroxide, acetyl peroxide, dilauryl peroxide, di-tert-butyl peroxide, cumyl hydroperoxide, hydrogen peroxide, or potassium persulfate may be used, and as the azo compound initiator, azo nitrile, azo ester, azo amide, azo imidazolin, azo amidine, or a macro azo initiator may be used. In the present invention, using an azo compound initiator, more preferably 4,4′-azobis(4-cyanovaleric acid), may be advantageous in terms of polymerization reaction efficiency.

In the present invention, after preparing a reverse phase emulsion, an initiator is added and reacted to obtain a crosslinked polymer having the viscosity desired in the present invention, that is, a thickener, and the yield of the thickener may be increased.

In one embodiment, the reaction temperature in step 1-3 may be 62° C. or higher, 65° C. or higher, 70° C. or higher, or 75° C., and the upper limit may be 100° C. The thickener may be prepared more stably in the above temperature range.

In one embodiment, step 1-3 may be performed for a reaction time of 1 to 12 hours, and preferably for 2 to 6 hours.

In another embodiment of the present invention, a method of preparing the amphiphilic thickener of the present invention may include:
dissolving a water-soluble monomer represented by Chemical Formula 1, a crosslinkable monomer, and a silicone-containing monomer represented by Chemical Formula 2 in an alcohol-based solvent (step 2-1);
raising a temperature to 60° C. or higher (step 2-2); and
adding a reaction initiator and performing polymerization to prepare a crosslinked polymer (step 2-3).

In one embodiment, as a method of preparing the amphiphilic thickener, in particular, precipitation polymerization may be used. The water-soluble monomer of Chemical Formula 1, the silicone-containing monomer of Chemical Formula 2, the crosslinkable monomer, and the reaction initiator used in the precipitation polymerization may be the same as those used in the PIT polymerization.

In one embodiment, the alcohol-based solvent used as the solvent may be an C₂₋₈ alcohol. A reaction yield may be further increased within the above range.

In the present invention, the solution itself in which the crosslinked polymer prepared by the above-described preparation method is formed may be used as a thickener. In addition, the solution may be precipitated with a precipitant to finally prepare a solid thickener such as powder. At this time, a precipitating non-solvent, especially, acetone may be used.

In addition, the present invention provides a thickener prepared by the above-described thickener preparation method.

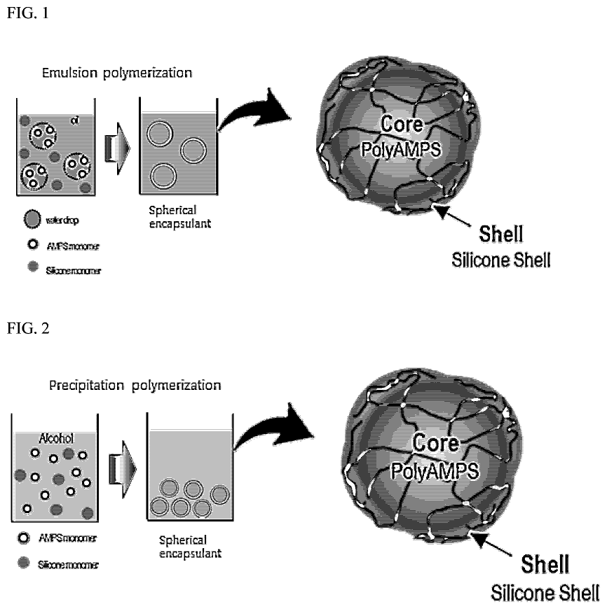

FIG. 1 is an exemplary diagram showing a method of preparing a polymer thickener (steps 1-1 to 1-3) according to the present invention.

The thickener according to the present invention may be in the form of a spherical thickener prepared in the same manner as shown in FIG. 1.

The thickener according to the present invention may be in the form of a spherical thickener prepared in the same manner as shown in FIG. 2.

The thickener according to the present invention has the form of spherical fine particles and may swell in water, ethanol, or a mixed solution thereof to provide a high-viscosity solution that is visually uniform in appearance.

The thickener prepared by the present invention may have an average particle diameter of 10 to 300 um.

In addition, the thickener prepared in the present invention may have a viscosity of 500 to 150,000 cPs or 5,000 to 100,000 cPs at 25° C. in a 1% (w/v) aqueous dispersion.

Hereinafter, the present invention will be described in more detail through examples and comparative examples. These examples are solely for illustrating the present invention, and the scope of the present invention is not to be construed as limited by these examples.

### EXAMPLES

### Comparative Example 1

Aqueous phase: 17 g of acrylamidomethylpropane sulfonic acid and 0.5 g of trimethylolpropane ethoxylate triacrylate were dissolved in 63 g of distilled water and then neutralized to pH 7-9 with ammonia water.

Oil phase: 10 g each of polyoxyethylene (3) oleyl ether and polyoxyethylene (6) oleyl ether were added to 80 g of heptane and mixed well.

The aqueous phase and the oil phase were placed in a reactor and heated to 70° C., 0.1 g of 4,4′-azobis(4-cyanovaleric acid) as an initiator was added and reacted for 3 hours, and the thickener was obtained by precipitation with acetone and drying.

### Comparative Example 2

Aqueous phase: 17 g of acrylamidomethylpropane sulfonic acid and 0.5 g of trimethylolpropane ethoxylate triacrylate were dissolved in 63 g of distilled water and then neutralized to pH 7-9 with ammonia water.

Oil phase: A certain amount of silicone methacrylate (molecular weight: 5,000) was added to 80 g of heptane and mixed well.

The aqueous phase and the oil phase were placed in a reactor and heated to 70° C., 0.1 g of 4,4′-azobis(4-cyanovaleric acid) as an initiator was added and reacted for 3 hours, and the thickener was obtained by precipitation with acetone and drying.

In this case, since the reaction performed without an emulsifier did not result in a uniform reaction and a gel was formed that was difficult to use for testing, emulsion polymerization using an emulsifier appears to be necessary.

### Examples 1 to 3

Aqueous phase: 17 g of acrylamidomethylpropane sulfonic acid and 0.5 g of trimethylolpropane ethoxylate triacrylate were dissolved in 63 g of distilled water and then neutralized to pH 7-9 with ammonia water.

Oil phase: A certain amount of silicone methacrylate (molecular weight: 1,000/5,000/10,000) was added to 80 g of heptane and then 10 g each of polyoxyethylene (3) oleyl ether and polyoxyethylene (6) oleyl ether were added and mixed well.

The aqueous phase and the oil phase were placed in a reactor and heated to 70° C., 0.1 g of 4,4′-azobis(4-cyanovaleric acid) as an initiator was added and reacted for 3 hours, and the thickener was obtained by precipitation with acetone and drying.

Each composition is shown in Table 1 below.

TABLE 1

| | | Comparative | Comparative | | | |
|---|---|---|---|---|---|---|
| | | Example | Example | Example | Example | Example |
| | | 1 | 2 | 1 | 2 | 3 |
| Aqueous | Distilled water | 63 | 63 | 63 | 63 | 63 |
| phase | Acrylamidomethyl | 17 | 17 | 17 | 17 | 17 |
| (units: | propane sulfonic | | | | | |
| g) | acid | | | | | |
| | Trimethylolpropane | 0.5 | 0.5 | 0.5 | 0.5 | 0.5 |
| | ethoxylate | | | | | |
| | triacrylate | | | | | |
| | Ammonia water | Neutralization | Neutralization | Neutralization | Neutralization | Neutralization |
| Oil | Heptane | 80 | 80 | 80 | 80 | 80 |
| phase | Silicone | — | — | 3.4 | — | — |
| (units: | methacrylate | | | | | |
| g) | (molecular weight: | | | | | |
| | 1,000) | | | | | |
| | Silicone | — | 3.4 | — | 3.4 | — |
| | methacrylate | | | | | |
| | (molecular weight: | | | | | |
| | 5,000) | | | | | |
| | Silicone | — | — | — | — | 3.4 |
| | methacrylate | | | | | |
| | (molecular weight: | | | | | |
| | 10,000) | | | | | |
| | Polyoxyethylene | 10 | — | 10 | 10 | 10 |
| | (3) oleyl ether | | | | | |
| | Polyoxyethylene | 10 | — | 10 | 10 | 10 |
| | (6) oleyl ether | | | | | |
| | 4,4′-Azobis(4- | 0.1 | | 0.1 | 0.1 | 0.1 |
| | cyanovaleric acid) | | | | | |

### Comparative Example 3

20 g of acrylamidomethylpropane sulfonic acid and 1 g of trimethylolpropane ethoxylate triacrylate were added to 100 g of t-butanol, and then 6.4 g of ammonia water was added and stirred for 30 minutes to completely dissolve them.

Thereafter, after raising a temperature to 70° C., 0.3 g of 4,4′-azobis(4-cyanovaleric acid) as an initiator was added and reacted for 3 hours, and the thickener was obtained by precipitation with ethanol and acetone and drying.

### Example 4

20 g of acrylamidomethylpropane sulfonic acid and 1 g of trimethylolpropane ethoxylate triacrylate were added to 100 g of t-butanol, and then 6.4 g of ammonia water was added and stirred for 30 minutes to completely dissolve them. Next, a certain amount of silicone methacrylate (molecular weight: 5,000) was added and mixed well.

Thereafter, after raising the temperature to 70° C., 0.3 g of 4,4′-azobis(4-cyanovaleric acid) as an initiator was added and reacted for 3 hours, and the thickener was obtained by precipitation with ethanol and acetone and drying.

Each composition is shown in Table 2 below (units: g).

TABLE 2

| | Comparative | |
|---|---|---|
| | Example 3 | Example 4 |
| t-Butanol | 100 | 100 |
| Acrylamidomethylpropane sulfonic acid | 20 | 20 |
| Trimethylolpropane ethoxylate triacrylate | 1 | 1 |
| Ammonia water | 6.4 | 6.4 |
| Silicone methacrylate (molecular weight: | — | 6 |
| 5,000) | | |
| 4,4′-Azobis(4-cyanovaleric acid) | 0.3 | 0.3 |

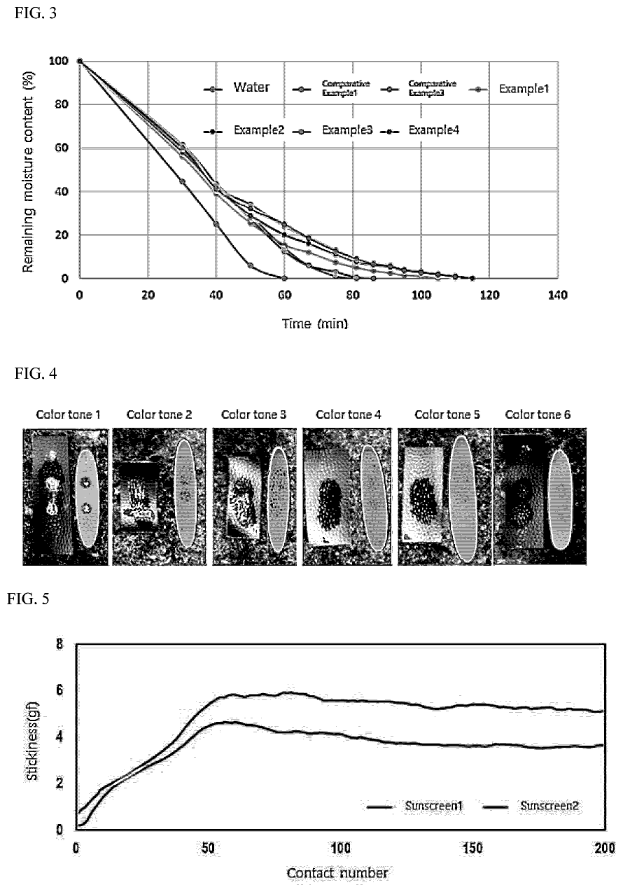

### Experimental Example 1. Comparison of Moisture Retention Capacity (FIG. 3)

1 g of each thickener prepared using the compositions of Comparative Examples 1 and 3 and Examples 1 to 4 was dissolved in 99 g of distilled water. Afterwards, 10 g of the polymer solution at 55° C. was transferred to a petri dish and placed in an oven. The weight of a sample was measured over time to confirm the amount of moisture evaporation. The results are shown in FIG. 3.

As a result, as shown in FIG. 3, the moisture retention time of the thickeners of Examples 1 to 4 was significantly increased compared to Comparative Examples 1 and 3. This means that when coated with silicone, moisture evaporation is suppressed in the moisture-containing core of the polymer, thereby increasing moisturizing power.

### Experimental Example 2. Comparison of Staining when Applying Color Cosmetics

First, color cosmetics formulations were prepared as shown in Table 3 below.

The raw materials corresponding to an oil phase were first mixed and melted by heating to 80° C. In the meantime, an aqueous phase was added and dissolved, and then the aqueous phase was added to the oil phase and homomixed at 5000 rpm for 10 minutes to prepare a formulation.

30 μl of the prepared formulation was dropped on the marble floor, spread thinly to a thickness of 25 μm, and dried for 4 hours. 10 μl of artificial sebum was dropped on the dried formulation, left at room temperature for 10 minutes, covered with artificial leather, and a force was applied by moving a 200 g weight back and forth 10 times. The covered artificial leather was removed and the amount of color cosmetics on the leather was visually compared. The results are shown in FIG. 4.

TABLE 3

| | Component name (units: | Color | Color | Color | Color | Color | Color |
|---|---|---|---|---|---|---|---|
| | g) | tone 1 | tone 2 | tone 3 | tone 4 | tone 5 | tone 6 |
| Aqueous | water | 24.5 | 22.5 | 22.5 | 22.5 | 22.5 | 22.5 |
| phase | Sodium polystyrene | 1 | 1 | 1 | 1 | 1 | 1 |
| | sulfonate | | | | | | |
| | 1,2-Hexanediol | 1.5 | 1.5 | 1.5 | 1.5 | 1.5 | 1.5 |
| | Comparative Example 1 | — | 2 | — | — | — | — |
| | Example 1 | — | — | 2 | — | — | — |
| | Example 2 | — | — | — | 2 | — | — |
| | Example 3 | — | — | — | — | 2 | — |
| | Example 4 | — | — | — | — | — | 2 |
| Oil | Cyclopentasiloxane | 30 | 30 | 30 | 30 | 30 | 30 |
| phase | Caprylic/capric | 6 | 6 | 6 | 6 | 6 | 6 |
| | triglyceride | | | | | | |
| | Silicone acrylate | 0.5 | 0.5 | 0.5 | 0.5 | 0.5 | 0.5 |
| | Diphenylsiloxyphenyltrimethicone | 2 | 2 | 2 | 2 | 2 | 2 |
| | Trimethylsiloxysilicate | 5 | 5 | 5 | 5 | 5 | 5 |
| | Lauryl PEG-9 | 3 | 3 | 3 | 3 | 3 | 3 |
| | polydimethylsiloxyethyl | | | | | | |
| | dimethicone | | | | | | |
| | Cetyl PEG/PPG-10/1 | 1 | 1 | 1 | 1 | 1 | 1 |
| | dimethicone | | | | | | |
| | Dimethicone/vinyl | 5 | 5 | 5 | 5 | 5 | 5 |
| | dimethicone | | | | | | |
| | crosspolymer | | | | | | |
| | Quaternium-18 hectorite | 0.5 | 0.5 | 0.5 | 0.5 | 0.5 | 0.5 |
| | Pigment No. 21 | 20 | 20 | 20 | 20 | 20 | 20 |

| | | | | | | |
|---|---|---|---|---|---|---|
| Total | 100 | 100 | 100 | 100 | 100 | 100 |

As shown in FIG. 4, it was confirmed that the polymer synthesized with a higher silicone methacrylate molecular weight showed less staining of color cosmetics. In cases where polymers were not used, color cosmetics had little sebum resistance, and even in the case of polymers that were not coated with silicone methacrylate, the color cosmetics had a high level of staining. This is believed to be because the silicone coated on the surface of the spherical polymer adheres closely to each other between the polymers and forms a film as the formulation is set, thereby exhibiting sebum resistance and water resistance.

### Experimental Example 3. Comparison of Stickiness when Applying Color Cosmetics

10 μl of each color cosmetic in Experimental Example 2 was dropped on artificial leather and spread evenly in an area of 2 cm×2 cm, and then stickiness according to drying time was measured. A cylindrical applicator was used as the texture analyzer, the measurement parameters were set to a measurement speed of 1 mm/s and a measurement force of 1.0 g, and the stickiness was calculated from the negative energy at the time the applicator first touched the dried formulation. The results are shown in Table 4 below.

TABLE 4

| | Color | Color | Color | Color | Color | Color |
|---|---|---|---|---|---|---|
| | tone 1 | tone 2 | tone 3 | tone 4 | tone 5 | tone 6 |
| Stickiness | 14.4 | 16.5 | 12.1 | 8.6 | 6.5 | 6.7 |
| (gf · s) | | | | | | |

As shown in Table 4, it was confirmed that when a general water-dispersed polymer was used, the stickiness was very strong, but when silicone methacrylate was used, the stickiness was less. This is because when water-dispersed polymers are used to address the dryness of color products, dryness decreases but stickiness increases, and this can be resolved using a spherical polymer coated with silicone methacrylate to reduce stickiness while maintaining moisture.

### Experimental Example 4. Comparison of Stickiness when Applying UV Protection Cosmetics

First, a UV protection cosmetics formulation was prepared as shown in Table 5 below.

The raw materials corresponding to an oil phase were first mixed and melted by heating to 80° C. In the meantime, an aqueous phase was added and dissolved, and then the aqueous phase was added to the oil phase and homomixed at 4000 rpm for 10 minutes to prepare a formulation.

3 μl of UV protection cosmetics (sunscreen) was dropped on the artificial leather, and then stickiness was measured according to the number of times the applicator touches it. A ball-type applicator was used as the texture analyzer, the measurement parameters were set to a measurement speed of 1 mm/s and a measurement force of 1.0 g, repeated 200 times, and stickiness was calculated from the negative energy at the time the applicator touched the formulation. The results are shown in FIG. 5.

TABLE 5

| | Component name | Sunscreen 1 | Sunscreen 2 |
|---|---|---|---|
| Oil phase | Cetyl ethylhexanoate | 2.00 | 2.00 |
| | Ethylhexyl salicylate | 20.00 | 20.00 |
| | Sorbitan olivate | 0.50 | 0.50 |
| | Hydrogenated polyisobutene | 2.00 | 2.00 |
| | Cetearyl alcohol | 2.00 | 2.00 |
| | Glyceryl stearate | 0.50 | 0.50 |
| | Cyclopentasiloxane | 10.00 | 10.00 |
| Aqueous | water | 53.68 | 54.08 |
| phase | Example 2 | — | 0.30 |
| | Tromethamine | 0.30 | 0.15 |
| | Dipropylene glycol | 5.00 | 5.00 |
| | 1,2-Hexanediol | 1.00 | 1.00 |
| | Cetyl phosphate | 2.5 | 2.5 |
| | Carbomer | 0.50 | 0.25 |
| | Trisodium EDTA | 0.02 | 0.02 |

| | | |
|---|---|---|
| Total | 100.0 | 100.0 |

As shown in FIG. 5, it was confirmed that when a general polymer was used, the stickiness was very strong, but when silicone methacrylate was used, the stickiness was less. This problem may be solved using a spherical polymer coated with silicone methacrylate to reduce stickiness while maintaining moisture in UV protection products, as in color products.

### Experimental Example 5. Comparison of Durability of UV Protection Performance when Applying UV Protection Cosmetics

33.5 μg of the sunscreen of Experimental Example 4 was thinly applied on a PMMA plate (Helioplate, Helioscreen, France) for measuring a UV protection index, the plate having a size of 5 cm×5 cm. After 15 minutes, an initial UV protection level (SPF) was measured using an in vitro measuring device, and 4 hours later, 10 μg of artificial sebum was dropped on the PMMA plate on which the sunscreen had been applied, spread evenly, and after 10 minutes, the SPF value was measured again to confirm the change in SPF. The results are shown in Table 6 below.

TABLE 6

| SPF | Initial | Later | Decrease rate (%) |
|---|---|---|---|
| Sunscreen 1 | 53.3 | 26.0 | 51.3 |
| Sunscreen 2 | 51.5 | 36.8 | 28.6 |

As shown in the above results, it was confirmed that when a spherical thickener coated with silicone methacrylate was used, the SPF reduction rate was lower than that of a sunscreen made using a general polymer. Similar to Experimental Example 2, it was found that the sebum resistance shown in color cosmetics was also applied to UV protection cosmetics, increasing the durability of UV protection performance.

### Experimental Example 6. Actual Use Test for Stickiness and Moisture when Applying Skin Formulation

First, a skin formulation for keratin management was prepared as shown in Table 7 below.

The skin formulation was applied to the faces of 20 women aged 25 to 50 once a day for 3 days. After application, a questionnaire asked about the feeling of use. Regarding each feeling of use, it was evaluated on the basis of “⊚: 16 or more out of 20 subjects said that it was not sticky or had high moisture, ∘: 12 to 15 out of 20 subjects said that it was not sticky or had high moisture, Δ: “8 to 11 out of 20 subjects said it was not sticky or had high moisture.” The results are shown in Table 7 below.

TABLE 7

| Component | Skin | Skin | Skin | Skin | Skin |
|---|---|---|---|---|---|
| name (units: g) | formulation 1 | formulation 2 | formulation 3 | formulation 4 | formulation 5 |
| water | To 100 | To 100 | To 100 | To 100 | To 100 |
| Dexpanthenol | 0.500 | 0.500 | 0.500 | 0.500 | 0.500 |
| 1,2-Hexanediol | 4.000 | 1.000 | 1.000 | 1.000 | 1.000 |
| Dipropylene glycol | 8.000 | 8.000 | 8.000 | 8.000 | 8.000 |
| Xanthan gum | 0.400 | 0.400 | 0.400 | 0.400 | 0.400 |
| Example 2 | — | 0.25 | 0.5 | — | — |
| Example 4 | — | — | — | 0.25 | 0.5 |
| Gluconolactone | 3.000 | 3.000 | 3.000 | 3.000 | 3.000 |
| Lactic acid | 3.000 | 3.000 | 3.000 | 3.000 | 3.000 |
| Total | 100.000 | 100.000 | 100.000 | 100.000 | 100.000 |
| Not sticky | Δ | ◯ | ⊚ | ◯ | ⊚ |
| Moisture | ◯ | ◯ | ⊚ | ◯ | ⊚ |

As may be seen from the results shown in Table 7, it was found that the keratin management ingredient in the formulation for managing keratin has high stickiness, but when the polymer provided in the present invention is used, stickiness is reduced and moisture is increased.

In the present invention, it is possible to provide a thickener in the form of homogeneous fine particles in which the core is composed of a water-soluble polymer crosslinker and the surface is coated with a silicone polymer. Therefore, the spherical particle-shaped thickener of the present invention has the advantage of high moisture retention due to the coated silicone polymer, which is the shell, while maintaining the shape of the particle even when swelling in moisture. In addition, the spherical particles, whose surface is coated with the silicone polymer, have high dispersibility in an oil phase, which helps to maintain makeup, and the spherical polymer has the effect of reducing stickiness and staining.

1. An amphiphilic thickener comprising:
a crosslinked polymer obtained by polymerizing a water-soluble monomer represented by the following Chemical Formula 1, a crosslinkable monomer, and a silicone-containing monomer represented by the following Chemical Formula 2: 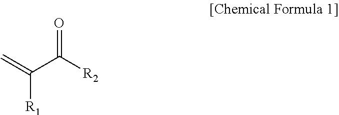
wherein
R₁ is —H, —CH₃, —CH₂CH₃, or —(CH₂)ₘCH₃, and here m=1 to 3,
R₂ is —OX, NH₂, or —NH—R₃—SO₃Y,
R₃ is —(CH₂)—, —(CH₂)₂—, —(CH₂)₃—, —(CH₂)₄—, —(CH₂)₂(CH₃)₂—, or —(CH₂)₃CH₃—, and
X and Y are H⁺, Na⁺, Li⁺, K⁺, or NH₃⁺ and may be the same, 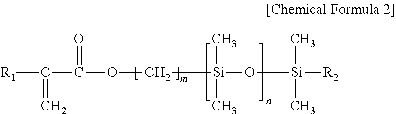
wherein
m is an integer from 1 to 5,
n is an integer from 10 to 5000.
R₁ is hydrogen or a C₁₋₃ alkyl group, and
R₂ is a hydroxyl group, a C₁₋₃ alkyl group, or a vinyl group.

2. The amphiphilic thickener of claim 1, wherein the water-soluble monomer represented by Chemical Formula 1 includes at least one selected from the group consisting of acrylamidomethylpropane sulfonic acid (AMPS), acrylic acid, acrylamide, and polyethylene glycol acrylate.

3. The amphiphilic thickener of claim 1, wherein the water-soluble monomer represented by Chemical Formula 1 is a compound represented by the following Chemical Formula 1a:

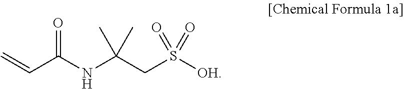

4. A method of preparing an amphiphilic thickener, comprising:
preparing an oil-in-water emulsion composition including an aqueous phase including a water-soluble monomer represented by the following Chemical Formula 1 and a crosslinkable monomer, and an oil phase including a silicone-containing monomer represented by the following Chemical Formula 2 and a non-polar organic solvent;
preparing a water-in-oil reverse phase emulsion composition by raising a temperature to 60° C. or higher; and
adding a reaction initiator and performing polymerization to prepare a crosslinked polymer, 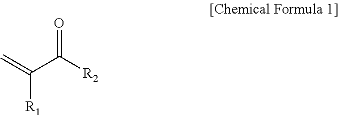
wherein
R₁ is —H, —CH₃, —CH₂CH₃, or —(CH₂)ₘCH₃, and here m=1 to 3,
R₂ is —OX, NH₂, or —NH—R₃—SO₃Y,
R₃ is —(CH₂)—, —(CH₂)₂—, —(CH₂)₃—, —(CH₂)₄—, —(CH₂)₂(CH₃)₂—, or —(CH₂)₃CH₃—, and
X and Y are H⁺, Na⁺, Li⁺, K⁺, or NH₃⁺ and may be the same, 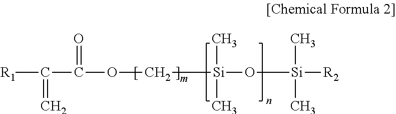
wherein
m is an integer from 1 to 5,
n is an integer from 10 to 5000,
R₁ is hydrogen or a C₁₋₃ alkyl group, and
R₂ is a hydroxyl group, a C₁₋₃ alkyl group, or a vinyl group.

5. A method of preparing an amphiphilic thickener, comprising:
dissolving a water-soluble monomer represented by the following Chemical Formula 1, a crosslinkable monomer, and a silicone-containing monomer represented by the following Chemical Formula 2 in an alcohol-based solvent;
raising a temperature to 60° C. or higher; and
adding a reaction initiator and performing polymerization to prepare a crosslinked polymer, 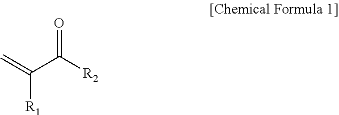
wherein
R₁ is —H, —CH₃, —CH₂CH₃, or —(CH₂)ₘCH₃, and here m=1 to 3,
R₂ is —OX, NH₂, or —NH—R₃—SO₃Y,
R₃ is —(CH₂)—, —(CH₂)₂—, —(CH₂)₃—, —(CH₂)₄—, —(CH₂)₂(CH₃)₂—, or —(CH₂)₃CH₃—, and
X and Y are H⁺, Na⁺, Li⁺, K⁺, or NH₃⁺ and may be the same, 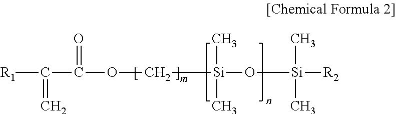
wherein
m is an integer from 1 to 5,
n is an integer from 10 to 5000,
R₁ is hydrogen or a C₁₋₃ alkyl group, and
R₂ is a hydroxyl group, a C₁₋₃ alkyl group, or a vinyl group.

6. The method of claim 4, wherein the water-soluble monomer represented by Chemical Formula 1 includes at least one selected from the group consisting of acrylamidomethylpropane sulfonic acid (AMPS), acrylic acid, acrylamide, and polyethylene glycol acrylate.

7. The method of claim 4, wherein the water-soluble monomer represented by Chemical Formula 1 is a compound represented by the following Chemical Formula 1a:

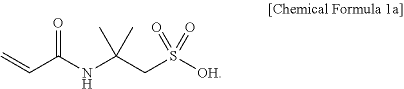

8. The method of claim 4, wherein the water-soluble monomer represented by Chemical Formula 1 is included in an amount of 50 to 95% by weight based on 100% by weight, which is the total sum of the water-soluble monomer represented by Chemical Formula 1, the crosslinkable monomer, and the silicone-containing monomer represented by Chemical Formula 2.

9. The method of claim 4, wherein the crosslinkable monomer is a compound having two or more acrylate groups, two or more acrylamide groups, or two or more vinyl groups.

10. (canceled)

11. The method of claim 4, wherein the silicone-containing monomer represented by Chemical Formula 2 is included in an amount of 5 to 50% by weight based on 100% by weight, which is the total sum of the water-soluble monomer represented by Chemical Formula 1, the crosslinkable monomer, and the silicone-containing monomer represented by Chemical Formula 2.

12. The method of claim 4, wherein the oil phase further includes one or more surfactants, and a total HLB value of the surfactant combination is 6 to 14.

13. The method of claim 12, wherein as the surfactant, two types of surfactants: a surfactant having an HLB value of 3 to 8; and a surfactant having an HLB value of 8 to 16 are used.

14. The method of claim 4, wherein the reaction initiator is one or more selected from the group consisting of peroxides and azo compounds.

15. (canceled)

16. (canceled)

17. The method of claim 4, further comprising performing precipitation with a precipitant to obtain a thickener in a solid form.
